(19) United States
(12) Patent Application Publication
(10) Pub. No.: US 2020/0113509 A1
(43) Pub. Date: Apr. 16, 2020

(54) Producing Output Data Relating to Skin Conditions

(71) Applicant: Zedsen Limited, London (GB)

(72) Inventor: Hrand Mami MAMIGONIANS, London (GB)

(21) Appl. No.: 16/595,697

(22) Filed: Oct. 8, 2019

(30) Foreign Application Priority Data
Oct. 11, 2018 (GB) 1816583.7

(51) Int. Cl. A61B 5/00

(52) U.S. Cl. CPC A61B 5/444; A61B 5/72; A61B 5/0064

(57) ABSTRACT

Output data is produced, indicative of a visible skin condition requiring medical attention. A reference region of skin that does not include the skin condition is identified and a probe is deployed against this reference region to produce reference data. The same probe is then re-deployed against the skin condition to produce test data. Output electrodes in the probe produce output signals by capacitive coupling when input electrodes receive energizing pulses. The reference data is derived from reference output signals and the test data is derived from test output signals. Output data is produced by comparing the test data against the reference data.

### CROSS REFERENCE TO RELATED APPLICATIONS

This application claims priority from United Kingdom Patent Application number 1816583.7, filed on Oct. 11, 2018, the whole contents of which are incorporated herein by reference.

### BACKGROUND OF THE INVENTION

The present invention relates to a method of producing output data indicative of a visible skin condition requiring medical attention.

It is known for trained clinicians to perform visual examinations upon areas of skin that appear abnormal. In particular, clinicians look to identify malignant conditions that contrast with benign conditions, given that the growth of a malignancy is not limited. Furthermore, a malignant condition is capable of invading into adjacent tissue and may be capable of spreading to distant tissues. Consequently, if a condition is identified as being potentially malignant, appropriate action may be required as a matter of urgency, due to the risks associated with increasing invasiveness and metastases.

Experiments have shown that cancerous cells possess different electrical and chemical properties compared to normal cells. It should therefore be possible to identify the presence of cancerous tissue by examining these electrical properties. However, a first problem arises in that invasive techniques may introduce undesirable medical complications. Furthermore, although differences occur due to the presence of skin cancer, differences also occur due to other factors and the differences due to the presence of skin cancer may be relatively modest. Consequently, a problem exists in terms of identifying variations due to the presence of skin cancer.

### BRIEF SUMMARY OF THE INVENTION

According to a first aspect of the present invention, there is provided an apparatus for producing output data indicative of a visible skin condition requiring medical attention, comprising: a manually-deployable probe; and a data processing system, wherein: said manually-deployable probe includes a plurality of electrodes, a strobing circuit and a monitoring circuit; said monitoring circuit monitors output signals from a selected output electrode produced by capacitive coupling when a selected input electrode receives an energizing strobing signal; and said data processing system is configured to: operate said probe to produce reference output signals; derive reference data from said reference output signals; operate said probe to produce test output signals; derive test data from said test output signals; and compare said test data against said reference data to produce said output data.

In an embodiment, the manually-operable probe includes a visual indicator, to indicate: an availability to be deployed; reference strobing; and test strobing. The manually operable probe may also include a manually operable device for indicating the start of reference strobing and the start of test strobing.

According to a second aspect of the present invention, there is provided a method of producing output data indicative of a visible skin condition requiring medical attention, comprising the steps of: identifying a reference region of skin that does not include said skin condition; deploying a probe against said reference region to produce reference data; and re-deploying said probe against said skin condition to produce test data, wherein: output electrodes produce output signals by capacitive coupling when input electrodes receive energizing pulses; said reference data is derived from reference output signals; said test data is obtained from test output signals; and output data is produced by comparing said test data against said reference data.

Embodiments of the invention will be described, by way of example only, with reference to the accompanying drawings. The detailed embodiments show the best mode known to the inventor and provide support for the invention as claimed. However, they are only exemplary and should not be used to interpret or limit the scope of the claims. Their purpose is to provide a teaching to those skilled in the art. Components and processes distinguished by ordinal phrases such as “first” and “second” do not necessarily define an order or ranking of any sort.

### BRIEF DESCRIPTION OF THE SEVERAL VIEWS OF THE DRAWINGS

FIG. 1 shows a graph of electrical characteristics;

FIG. 2 illustrates apparatus embodying the present invention;

FIG. 3 shows a matrix of electrodes of the type included in the probe identified in FIG. 2;

FIG. 4 shows the attachment of a dielectric laminate onto printed circuit boards;

FIG. 5 shows the connection of circuit boards identified in FIG. 4 onto a base circuit board;

FIG. 6 shows a schematic representation of a probe;

FIG. 7 shows an example of an energizing circuit;

FIG. 8 shows an example of an analog processing circuit;

FIG. 9 shows an example of a multiplexing environment;

FIG. 10 details the probe identified in FIG. 2;

FIG. 11 shows a method embodying an aspect of the present invention;

FIG. 12 details procedures for producing reference signals, as identified in FIG. 11;

FIG. 13 details procedures for producing two-dimensional signals identified in FIG. 12;

FIG. 14 shows an example of a visual representation;

FIG. 15 illustrates forward layering;

FIG. 16 illustrates reverse layering;

FIG. 17 illustrates sophisticated multi-combinational layering;

FIG. 18 illustrates the generation of reference output signals;

FIG. 19 illustrates operations performed to identify a reference peak and a reference interval;

FIG. 20 illustrates strobing operations within a scan cycle;

FIG. 21 illustrates the sampling of an output signal; and

FIG. 22 illustrates comparisons made between test data and reference data.

### DETAILED DESCRIPTION OF EMBODIMENTS OF THE INVENTION

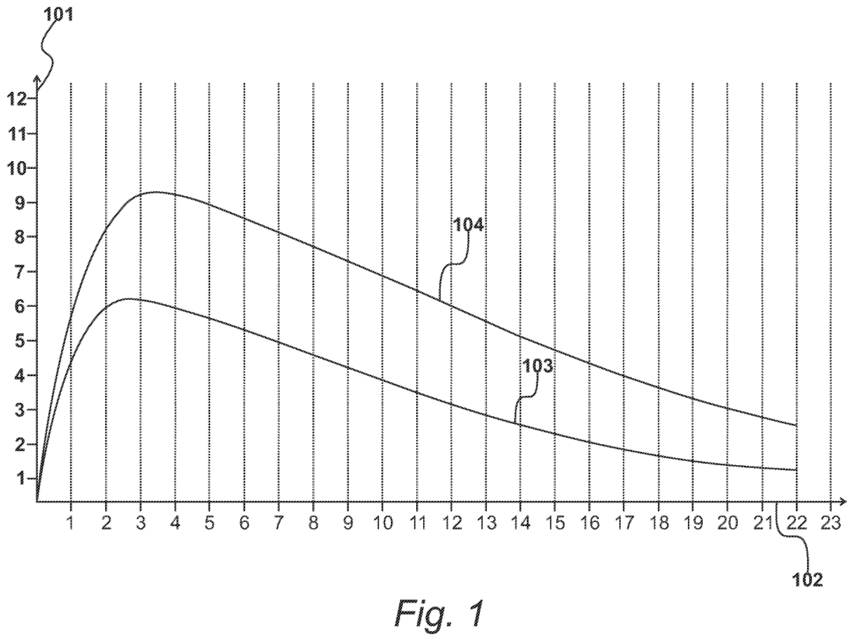

### FIG. 1

Many visible skin conditions exist that are benign and as such do not require further medical attention. However, existing visible skin conditions may instigate the start of a more serious condition and serious conditions themselves may exhibit new visible indications. Thus, demand exists for a technical solution that is capable of producing output data indicative of a visible skin condition requiring medical attention.

Many skin conditions that require medical attention will be identified as skin cancers, that are due to the development of abnormal cells that have the ability to spread to other parts of the body. In particular, three main types of skin cancer are known, usually identified as basal-cell skin cancer, squamous-cell skin cancer and melanoma. Melanomas are the most aggressive form of skin cancer and early signs include existing moles that have changed in size, shape or color.

The present inventor has conducted experiments on skin tissue with known cancerous cells. These experiments have involved capacitively coupled electrodes that form an electric field that extends into the skin tissue, thereby allowing electrical characteristics to be determined; primarily, the electrical permittivity and the electrical conductivity of the tissue.

Initial investigations deployed equipment substantially similar to that disclosed in U.S. Pat. No. 8,994,383 assigned to the present applicant. An illustration of the results obtained is shown in FIG. 1, in which the voltage of an output signal, is shown plotted on a first axis 101, against time measured in microseconds plotted against a second axis 102. The results for normal skin cells are shown at 103, with similar results for cancerous cells shown at 104. From this, it is clear that the interaction of an electric field with cancerous tissue is different from the interaction of a similar field with normal tissue. In particular, cancerous cells possess different electrical and chemical properties compared with normal cells and this is reflected in the resulting measurement of electrical properties.

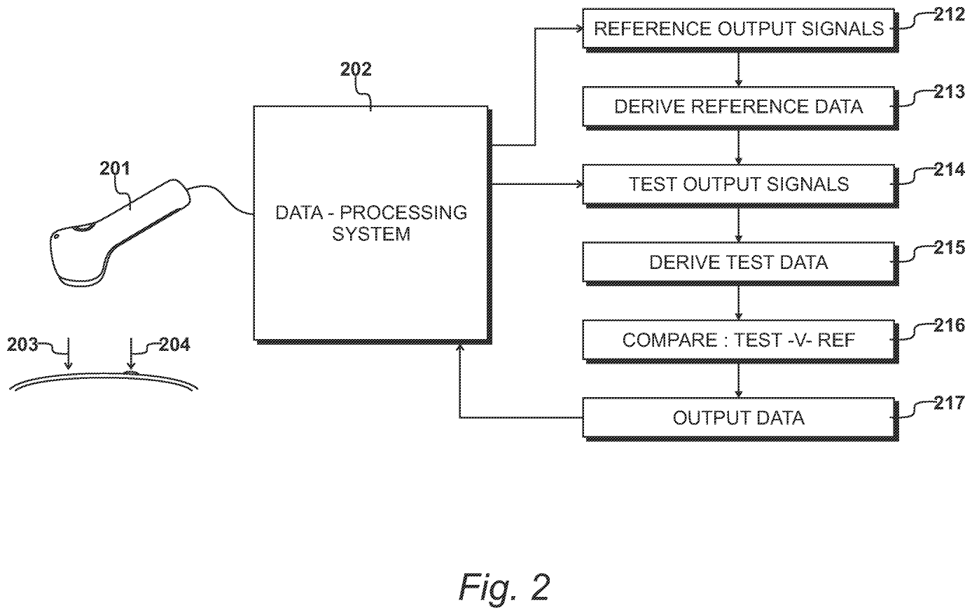

### FIG. 2

The present invention provides an apparatus for producing output data indicative of a visible skin condition requiring medical attention. The apparatus provides a manually deployable probe 201 and a data processing system 202. Two difficulties arise in terms of deploying this apparatus. Firstly, accurate data is required in relation to the tumour tissue and in this respect, accurate positioning of the probe becomes important. In addition, an appropriate reference is required to make an accurate assessment to the effect that the measurements obtained relate clearly to a skin condition that requires medical attention or a skin condition that may be considered benign.

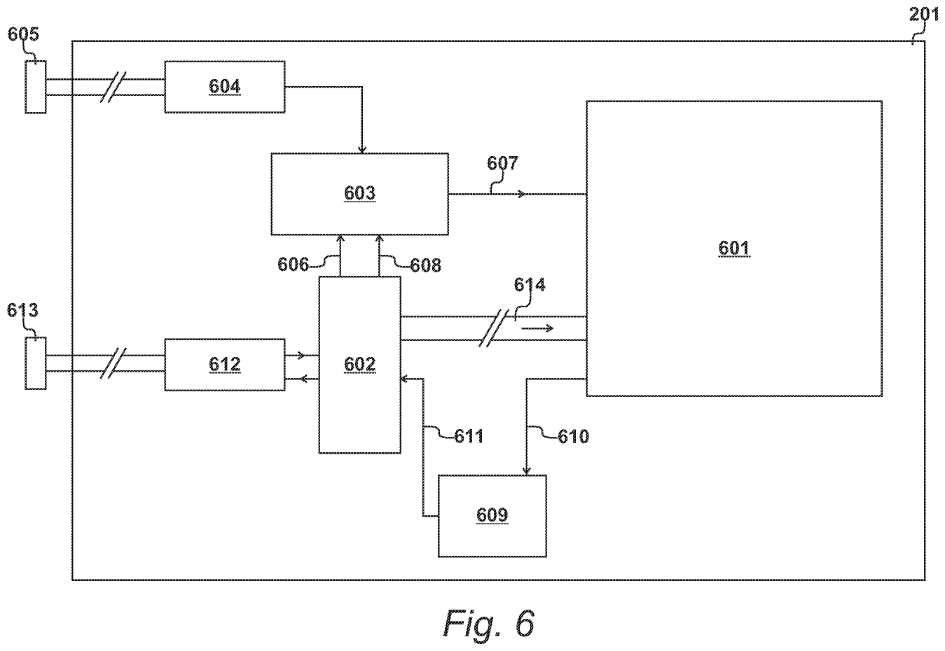

The probe includes a plurality of electrodes, a strobing circuit and a monitoring circuit, described in detail with reference to FIGS. 6 to 9. The monitoring circuit receives output signals, from a selected output electrode, produced by capacitive coupling when a selected input electrode receives an energizing strobing signal. In terms of addressing the first problem above, visual examination is required to ensure that, when producing test signals, the probe is correctly aligned over the visible skin condition. In a first embodiment, this may be achieved by the patient themselves, or by a trained clinician. In alternative embodiments, image recognition systems may be deployed to identify an appropriate location. In this way, image detection made by the image recognition system may be compared against image data produced by an embodiment of the present invention.

Further disclosure herein will relate to the manually deployed probe, with an option existing for graphical information to be displayed representing the region of the visible skin condition. Furthermore, as disclosed herein, further sophisticated techniques are then deployed to produce output data in the form of a recommendation as to whether further medical attention is required. Thus, as a result of a positive outcome, it may become necessary to conduct a physical biopsy. However, an aim of the present invention is to avoid unnecessary biopsies of this type being taken and thereby minimize unnecessary surgical intervention.

It is known, with apparatus of this type, to conduct a calibration procedure before the apparatus is used. An initial calibration of this type may be undertaken with respect to the present embodiment, in which an assessment is made of the electrical properties of the surrounding atmosphere. However, further experiments suggest that a simple calibration of this nature does not provide sufficient resolution when making assessments concerning the assessment of cancerous skin conditions. Thus, the present embodiment addresses the second problem in terms of providing greater accuracy when identifying skin conditions that do require medical attention.

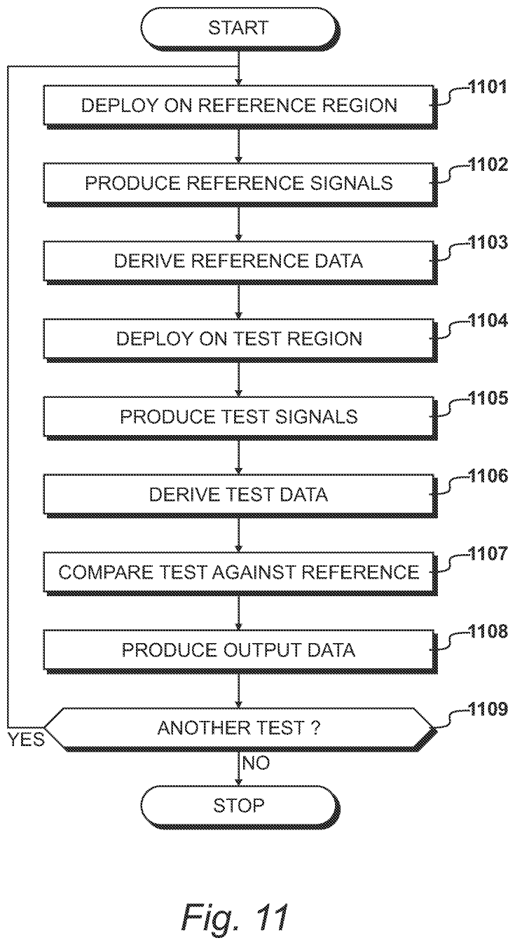

In particular, specific reference output signals are produced with the probe deployed against a region of normal skin. From these reference output signals, reference data is derived. The same probe is then operated again to produce test output signals, with the probe deployed against the visible skin condition. From these test signals, test data is derived. Thereafter, the test data is compared against specific reference data to produce output data. In this way, the accuracy of the assessment is significantly enhanced. Furthermore, in embodiments disclosed with reference to FIG. 11 onwards, sophisticated analysis techniques are performed to optimize the way in which the comparison of data takes place; again, with a view to increasing the overall accuracy of the tests being performed.

As shown in FIG. 2, the data processing system 202 configures the probe 201 to produce reference output signals, at stage 212, derived from a normal area of skin, as indicated by arrow 203. Reference data is then derived, at stage 213 from the reference output signals.

The same probe 201 (or in an alternative embodiment, a similar probe) is then deployed against the visible skin condition, as indicated by arrow 204. Thus, at stage 214, test output signals are produced, from which test data is derived at stage 215.

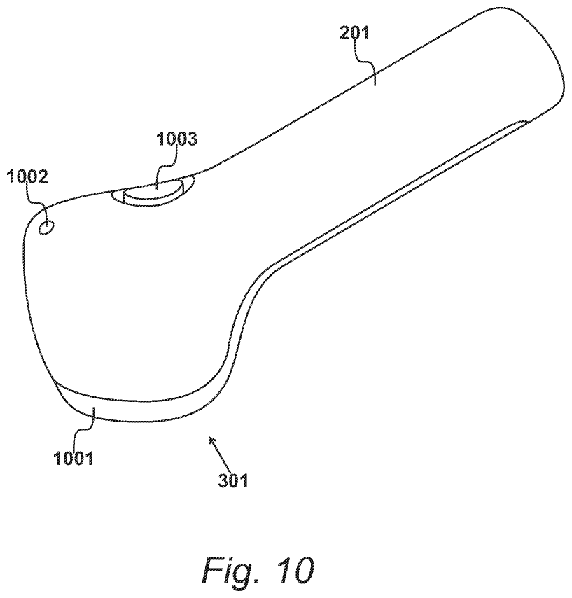

The data processing system 202 is then configured to perform a comparison between the test data derived at stage 215 and the reference data derived at stage 213, to produce the output data at stage 217. As described with reference to FIG. 10, further visual indications are generated by the probe to assist in completing this procedure.

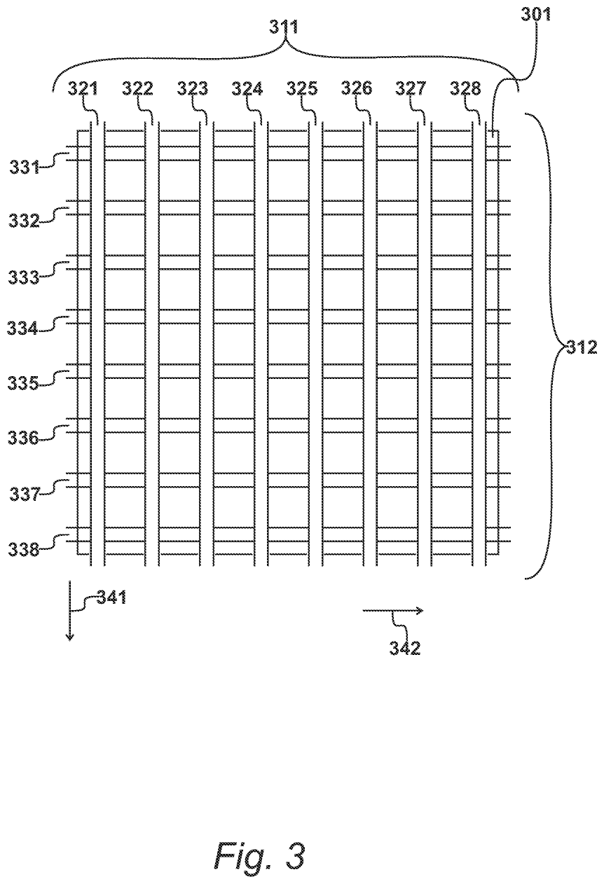

### FIG. 3

The probe 201 includes a matrix of electrodes, as illustrated in FIG. 3. The matrix of electrodes is supported on a dielectric laminate 301, upon which a first electrode set 311 is mounted on an upper surface and a second electrode set 312 is mounted on a lower surface. The first electrode set 311 (shown vertically in FIG. 3) consists of a first upper electrode 321 to an eighth upper electrode 328. Similarly, the second electrode set 312 comprises a first lower electrode 331 to an eighth lower electrode 338. Alternative embodiments may have more of fewer electrodes in either or both of the electrode sets.

The probe 201 produces output signals by capacitive coupling. Thus, output electrodes produce output signals when input electrodes receive energizing pulses. A period of time is therefore required between initiating the generation of the energizing pluses (also referred to as strobing) and completing the sampling of the resulting output signals from the output electrodes. The procedures performed during this interval collectively define a strobing operation and many operations of this type are performed within each scanning cycle.

Thus, each scanning cycle represents a particular selection of input electrodes (transmitter electrodes) and output electrodes (receiver electrodes). If necessary, a particular strobing operation may be repeated and, as disclosed herein, in an embodiment, repeats of this nature are required when producing reference data. Similarly, having completed a scanning cycle, the whole cycle may be repeated and the number of repeats may be related to the complexity of the cycle itself, particularly in terms of the number of strobing operations performed within each scanning cycle and the extent to which each strobing operation must be repeated.

In the current embodiment, three different types of scanning cycle are deployed. These consist of two-dimensional scanning, vertical scanning and horizontal scanning. Two-dimensional scanning is performed to obtain a visual image of a test area. This may assist operatives and clinicians and may enable a historical record of the condition to be retained. However, in this embodiment, the actual test itself relies upon vertical scanning and horizontal scanning techniques.

Two-dimensional scanning involves selecting a transmitter electrode from one of the electrode sets, with the receiver electrode being selected from the other electrode set. All possible strobing combinations are then performed. This may then represent the end of the scanning cycle but, in an embodiment, the scanning cycle then involves similar procedures being performed with the rolls of the transmitter set and receiver set being reversed. Thus, each available electrode within the matrix is selected as a transmitter electrode and as a receiver electrode, provided that the same electrode is not selected for performing both operations.

When performing two-dimensional scanning, the first upper electrode 321 may be selected as a transmitter electrode, with the first lower electrode 331 being selected as the receiver electrode, for the first strobing operation. Thereafter, a second strobing operation is performed, with the first upper electrode maintained as the transmitter electrode but with the second lower electrode selected as the receiver electrode. Thus, the strobing operations continue with each of the electrodes of the second electrode set 312 being selected as a receiver. Thereafter, the second upper electrode 322 is selected as the transmitter and again each electrode of the second electrode set is selected as a receiver, from the first lower electrode 331 to the eighth lower electrode 338.

Eventually, all possible combinations will have been considered to complete the scanning cycle, resulting in a two-dimensional array of values representing the underlying electrical characteristics of the skin condition. However, at this stage, it should be appreciated that these merely provide a visual representation which could, as an alternative, be achieved using standard photographic techniques.

Vertical scanning involves a scanning procedure in the vertical direction, that is, in the direction of arrow 341. For this, only the second electrode set 312 is used. In the vertical scanning cycle, a first strobing operation may consist of energizing the first lower electrode 331 as a transmitter electrode and monitoring the second lower electrode 332 as the receiver electrode. This only creates a one-dimensional scan but a significant region of the material experiences the resulting electric field. When generating reference data, in an embodiment and as described later, multiple strobing operations are performed to determine a maximum peak value for the resulting output signals. Furthermore, when generating test data, it is not necessary to perform multiple strobing operations but multiple samples of the resulting output signals are taken. Thus, similar patterns of strobing operations are performed when generating reference data and when generating test data but the actual nature of the individual strobing operations are different, in an embodiment, to achieve an improved level of optimization.

In an embodiment, a process identified by the inventor as “layering” is performed, which results in deeper degrees of penetration being achieved, thereby allowing an evaluation to be made as to the extent to which the skin condition has penetrated into the lower layers of the skin. Thus, the first lower electrode 331 may be energized again but deeper penetration is achieved by monitoring the third lower electrode 333. Further depth is achieved by monitoring the fourth lower electrode 334, followed by the fifth 335, the sixth 336, the seventh 337 and the eighth 338. Thus, deeper levels of penetration are obtained using this layering technique and, in an embodiment, a higher degree of energization may be deployed with increasing depth.

Thus, forward layering consists of selecting receiver electrodes in the direction of arrow 341. In addition to this, receiver electrodes in the opposite direction are also selected to produce data using reverse layering.

In an embodiment, it is possible for only the first lower electrode 331 to be selected as a transmitter for forward layering, with only the eighth lower electrode 338 being selected as a transmitter for reverse layering. However, in an embodiment, forward layering and reverse layering are performed with each available electrode being selected as a transmitter. The inventor describes this technique as “dynamic layering”.

Having completed vertical scanning as disclosed above, similar techniques are deployed for horizontal scanning, in which strobing operations progress in the direction of arrow 342. Thus, it can be appreciated that a significant data set is derived using a vertical scanning procedure followed by a horizontal scanning procedure. Furthermore, using the techniques of layering and reverse layering, it is possible to penetrate deeper into the skin, such that an assessment can be made as to the extent to which medical attention may be required. Furthermore, it should also be understood that the sophisticated layering techniques are deployed with respect to both the generation of reference data and test data, such that each individual data point in each data set may be compared against a corresponding point in the complementary set. In this way, multiple comparisons may be made and a final output may be derived based on an average or upon a more sophisticated statistical assessment.

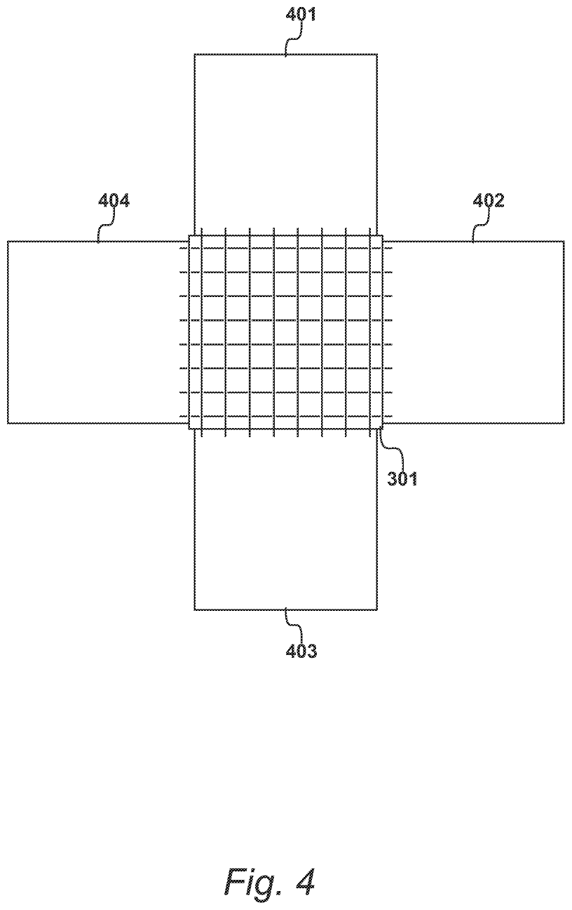

### FIG. 4

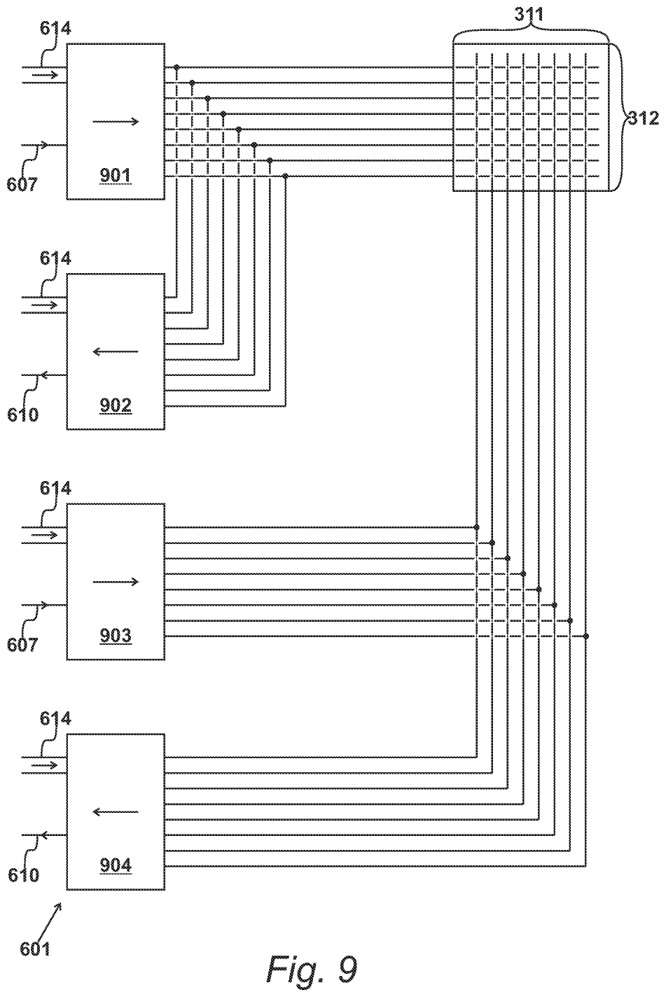

In an embodiment, the dielectric laminate 301 is supported by a first circuit board 401, a second circuit board 402, a third circuit board 403 and a fourth circuit board 404. In this way, electrodes of the first electrode set 311 are connected between the first circuit board 401 and the third circuit board 403, with electrodes of the second electrode set 312 being connected between the fourth circuit board 404 and the second circuit board 402. Multiplexing devices, described with reference to FIG. 9, are attached to these supporting circuit boards 401 to 404.

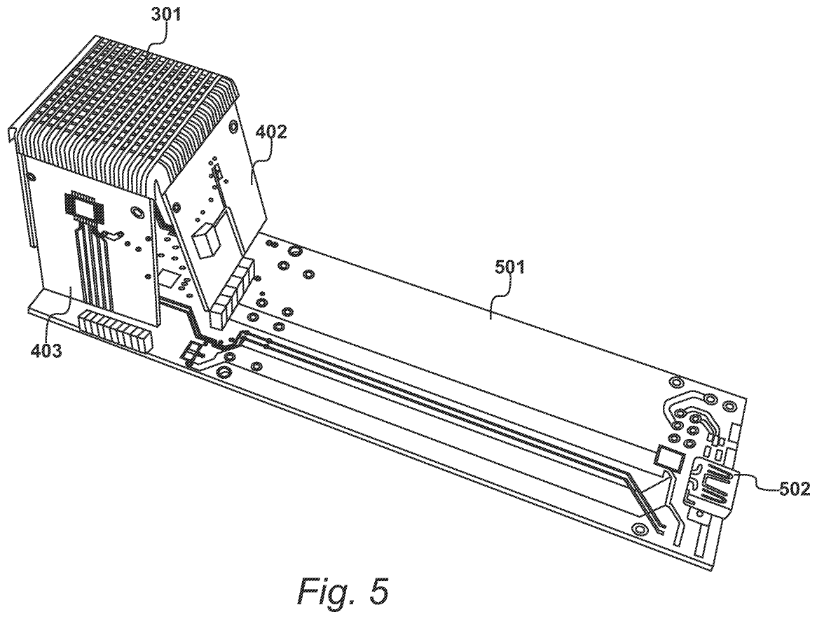

### FIG. 5

As shown in FIG. 5, in an embodiment, the supporting circuit boards 401 to 404 are folded and then secured to a base circuit board 501. Further circuitry, described with reference to FIGS. 6 to 8, are supported on the base circuit board 501, which also includes a standard USB socket 502, providing communication with the data processing system 202. Alternative embodiments may use alternative data communication protocols, possibly using radio transmission.

### FIG. 6

A schematic representation of the probe 201 is illustrated in FIG. 6. The matrix of electrodes on the dielectric membrane is included within a multiplexing environment 601. The multiplexing environment 601 also includes de-multiplexers for selectively de-multiplexing multiplexed energizing input voltage pulses for application to each of the electrodes, along with multiplexers for selectively multiplexing output signals monitored from each of the electrodes.

A microcontroller 602 controls the de-multiplexers and the multiplexers to ensure that the same electrode cannot both be energized and monitored during the same strobing operation.

An energizing circuit 603 is energized by a power supply 604 that in turn may receive power from an external source via a power-input connector 605. A voltage-control line 606, from a digital-to-analog convertor within the microcontroller 602, to the energizing circuit 603 allows the microcontroller 602 to control the voltage (and hence energy) of energizing signals supplied to the multiplexing environment 601, via a strobing line 607. The timing of each strobing signal is controlled by the microcontroller 602 via a trigger-signal line 608.

An output from the multiplexing environment 601 is supplied to an analog processing circuit 609 over a first monitoring line 610. A conditioning operation is performed by the analog processing circuit 609, allowing analog output signals to be supplied to the microcontroller 602 via a second monitoring line 611. The processor 602 also communicates with a two-way data-communication circuit 612, thereby allowing a data interface 613 to connect with the data processing system.

In operation, the microcontroller 602 supplies addresses over an address bus 614 to the multiplexing environment 601. Thus, having supplied addresses to the multiplexing environment 601, a strobing voltage is supplied via strobing line 607, resulting in an output signal being supplied to the processor 602. At the processor 602, an analog input signal is sampled and digitally encoded.

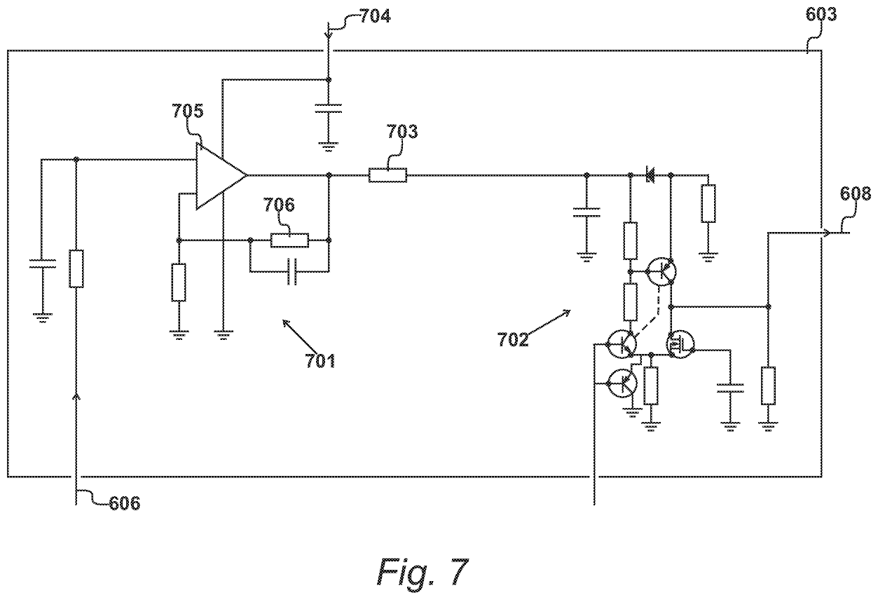

### FIG. 7

An example of the energizing circuit 603 is shown in FIG. 7. The energizing circuit 603 consists of a voltage-control circuit 701 connected to a strobing circuit 702 via a current-limiting resistor 703.

A voltage input line 704 receives energizing power from the power supply 604 to energize an operational amplifier 705. The operational amplifier 705 is configured as a comparator and receives a reference voltage via feedback resistor 706. This is compared against a voltage-control signal received on the voltage-control line 606 to produce an input voltage for the strobing circuit 702.

In the embodiment of FIG. 11, the strobing circuit 702 includes two bipolar transistors configured as a Darlington pair, in combination with a MOSFET. This creates strobing pulses with sharp rising edges and sharp falling edges that are conveyed to the strobing line 608.

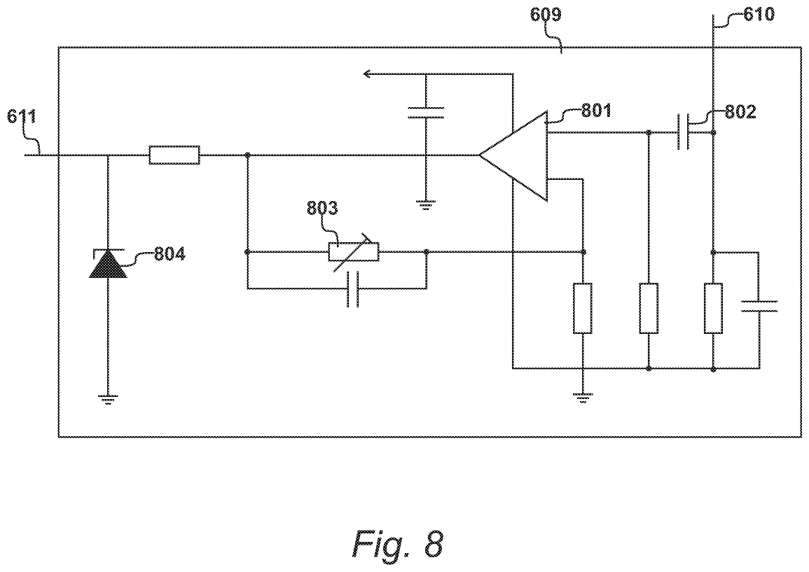

### FIG. 8

An example of an analog processing circuit 609 is illustrated in FIG. 8. Signals received on the first monitoring line 610 are supplied to a buffering amplifier 801 via a decoupling capacitor 802. During an initial set-up procedure, a variable feedback resistor 803 is trimmed to optimize the level of monitored signals supplied to the processor 602 via the second monitoring line 611. A Zener-diode 804 prevents excessive voltages being supplied to the processor 602.

### FIG. 9

An example of the multiplexing environment 601 is detailed in FIG. 9, that includes a first multiplexer 901, a second multiplexer 902, a third multiplexer 903 and a fourth multiplexer 904, each addressed by a portion of the address bus 614.

The first multiplexer 901 provides a de-multiplexing function and supplies input signals to selected transmitter electrodes of the first set 311. The second multiplexer receives output signals from the first set 311. Similarly, the third multiplexer supplies input signals to the second set 312 and the fourth multiplexer receives output signals from the second set 312. In this way, every available electrode may be selected as a transmitter or as a receiver.

### FIG. 10

Probe 201 is detailed in FIG. 10. The dielectric laminate 301 is surrounded by a protruding ring 1001 that is configured to be brought into contact with a patient's skin when the probe is deployed. The protruding ring 1001 extends slightly beyond the plane of the dielectric laminate such that, when pushed against a patient's skin, the dielectric laminate is brought into contact with the skin (the upper electrodes having an insulating layer applied over them) to maintain a constant degree of applied pressure when multiple deployments are made.

In this embodiment, the probe 201 includes a light-emitting device 1002 and a manually-activated button 1003. The light-emitting device 1002 is activated by the data processing system 202, to emit light of different colors. In an embodiment, the light emitting device initially emits red light to indicate a ready condition. The probe is then deployed to perform a reference scan by being placed upon an area of skin that does not exhibit the visible skin condition. While maintaining deployment of the probe in this position, button 1003 is pressed and a reference scan is performed. While the reference scan is being performed, the light-emitting device emits a flashing blue light which continues until the reference scan has been completed.

Upon completion of a reference scan, the light-emitting device 1002 again emits red light, indicating that a further application of the manual button 1003 is required. This time, the probe is deployed at the position of the visible skin condition and, following the application of appropriate pressure, button 1003 is pressed again. On this occasion, in this embodiment, the light-emitting device 1002 is energized to flash green, indicating that a test scan is being performed. Eventually, the test scan will complete and the light emitting device 1002 will again emit red light.

Upon achieving the red condition, the overall process may be repeated again on the same patient, possibly with a different skin condition being selected for the production of test data. Thereafter, the protruding ring 1001 may be detached, such that components making physical contact with patients are only deployed on a single use basis.

It can be appreciated that other embodiments may deploy alternative interface devices for informing an operative as to the operating state of the probe.

### FIG. 11

The apparatus described with reference to FIGS. 1 to 10 facilitates the adoption of a method of producing output data indicative of a visible skin condition requiring medical attention. The method comprises the steps of identifying a reference region of skin that does not include the skin condition, followed by deployment of the probe against the reference region to produce reference data. Thereafter, in this embodiment, redeployment of the same probe is made against the skin condition itself, to produce test data.

Output electrodes produce output signals by capacitive coupling when input electrodes receive energizing pulses. In this way, it is possible for the reference data to be derived from reference output signals and the test data to be obtained from test output signals. Output data is then produced by comparing the test data against the reference data. Consequently, significant accuracy enhancements are achieved, given that a comparison has been made between an area of skin that includes the visible skin condition and an area of skin, on the same patient, that does not include the visible skin condition.

In an embodiment, the method is achieved under the control of the separate data processing system 202. However, in alternative embodiments, all of the processing capability could be contained within the probe itself or, alternatively, communication could take place wirelessly with a wireless device such as a mobile cellular telephone. In addition, a mobile cellular telephone configured to operate in this way could in turn communicate with remote database systems.

In a preferred method, as illustrated in FIG. 11, the probe is deployed on a reference region at step 1101. Thereafter, at step 1102, reference signals are produced and at step 1103 reference data is derived from the reference signals.

At step 1104, the same probe is deployed on a test region, to produce test signals at step 1105. Test data is derived at step 1106, from the test signals produced at step 1105. Thereafter, the test data is compared against the reference data at step 1107, to produce output data at step 1108. Following this, at step 1109, a question is asked as to whether another test is to be performed. Thus, a further test could be performed upon the same test region (that is, the same visible skin condition causing concern) or a different reference region could be selected, thereby providing greater reassurance as to the accuracy of the test results.

Alternatively, a different visible skin condition on the same patient could be considered or, following appropriate hygiene requirements, similar tests could be performed on a different patient until, eventually, the question asked at step 1109 is answered in the negative.

In an embodiment, two-dimensional scanning, vertical scanning and horizontal scanning are all performed for the reference region 203 and the test region 204. Like-for-like comparisons are then made at step 1107, from which a visual representation may be derived based on the two-dimensional scan data, followed by a diagnosis based on the vertical and horizontal scan data.

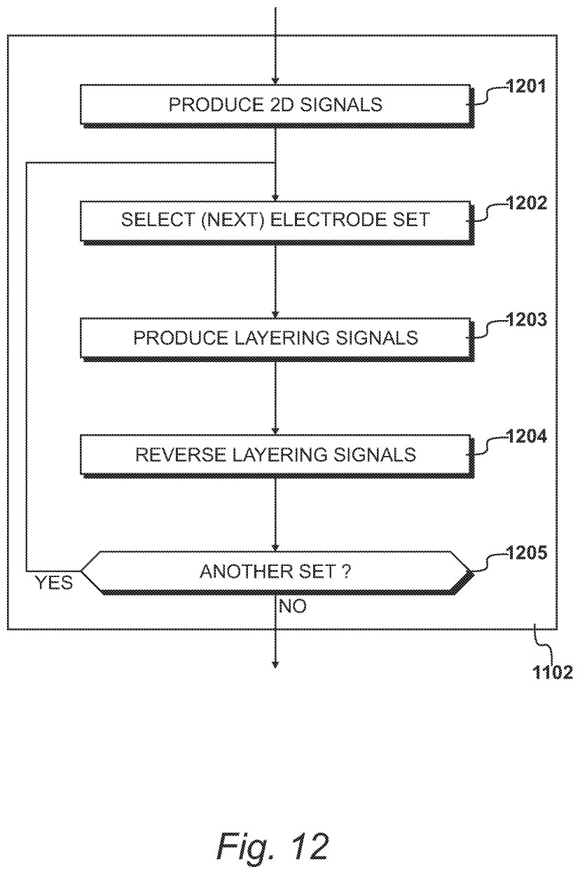

### FIG. 12

Procedures 1102 for producing reference signals are detailed in FIG. 12. These procedures are substantially similar to those performed at step 1105 to produce test signals, with specific differences that will be detailed herein.

At step 1201, two-dimensional scan signals are produced by the two-dimensional scanning technique previously described. In an embodiment, the first electrode set is selected for transmission and the second electrode set is selected for reception. In this embodiment, this provides sufficient data for a visual representation to be established, particularly given that two-dimensional scanning is not used, in this embodiment, for the purposes of diagnosis.

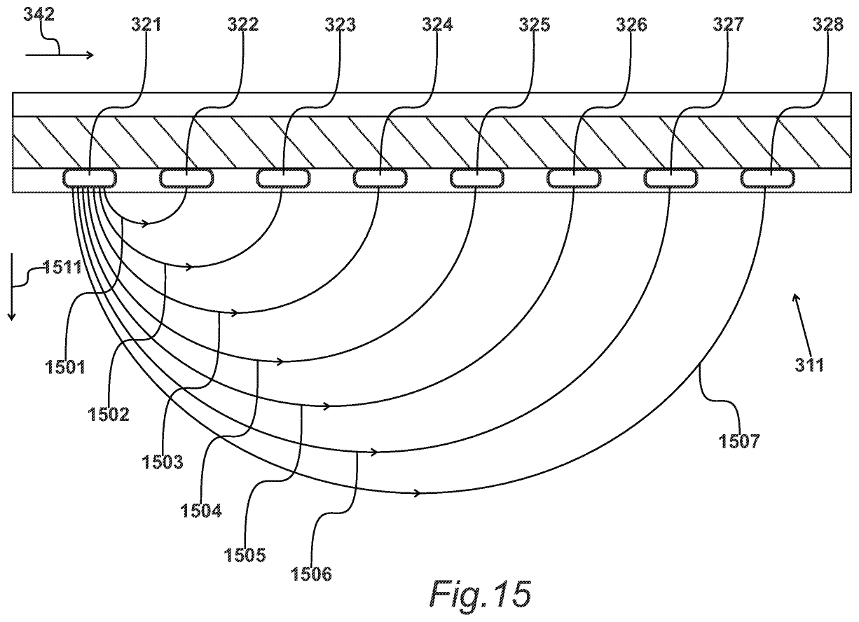

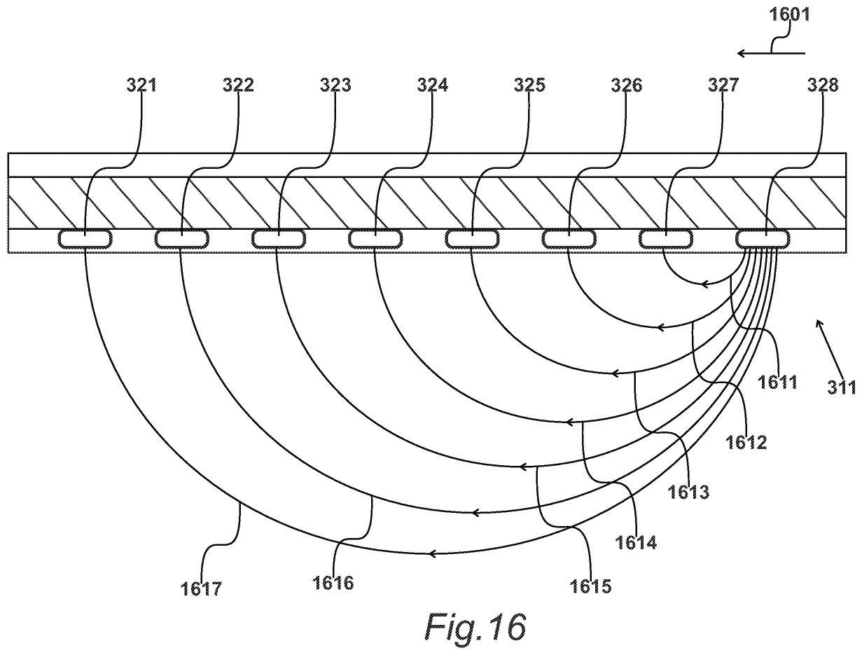

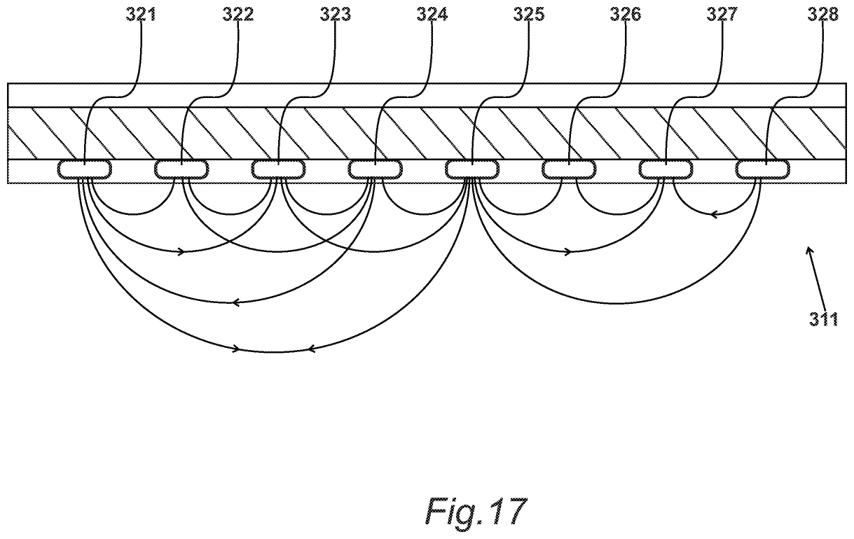

Vertical scanning is then performed by selecting an electrode set at step 1202. After an electrode set has been selected at step 1201, layering signals are produced at step 1203. In this embodiment, this is then followed by the production of reverse layering signals at step 1204. Thus, in an embodiment, forward layering is conducted, as described with reference to FIG. 15, followed by reverse layering, as described with reference to FIG. 16. Alternatively, forward and reverse layering techniques may be combined and the overall extent of layering may be increased, possibly including all possible combinations of transmitter electrode and receiver electrode within each of the two planes, as described further with reference to FIG. 17.

After completing the forward layering technique and the reverse layering technique, a question is asked at step 1205 as to whether another electrode set is to be considered which, on the first iteration, will be answered in the affirmative, resulting in the next electrode set being selected at step 1202. Thus, on the second iteration, the second electrode set 312 is selected and similar procedures are conducted for the production of forward-layering signals and reverse-layering signals.

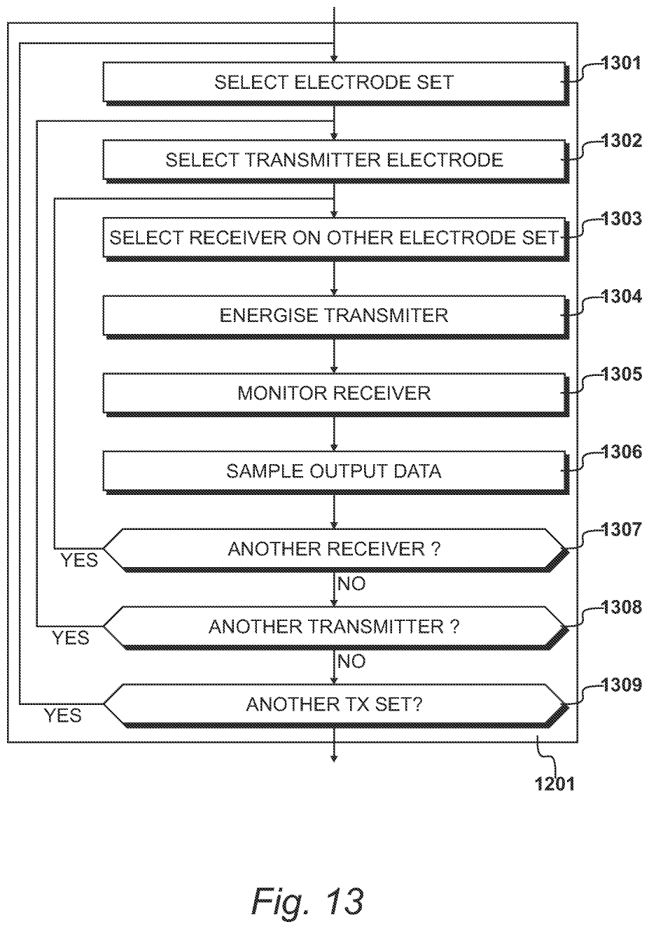

### FIG. 13

Procedures 1201 for producing two-dimensional signals are illustrated in FIG. 13. At step 1301, an electrode set is selected which, for the purposes of this illustration, may be the first electrode set 311. At step 1302, a transmitter electrode is selected from the first electrode set, such as the first upper electrode 321. Thereafter, a receiver electrode is selected from the second electrode set 312 which could be the first lower electrode 331.

At step 1304, the transmitter electrode is energized and at step 1305, the receiver electrode is monitored. Output data is sampled at step 1306 and at step 1307 a question is asked as to whether another receiver is to be considered. Thus, when answered in the affirmative, the next receiver electrode is selected (the second lower electrode 332) and the process is repeated. Eventually, all of the receiver electrodes will have been selected and the question asked at step 1307 will be answered in the negative.

At step 1308, a question is asked as to whether another transmitter electrode is available and when answered in the affirmative, the next transmitter electrode is selected at step 1302 which, in this example, is the second upper electrode 322. Thus, all of the receiver electrodes are monitored for this second selected transmitter electrode, resulting in the question asked at step 1307 being answered in the negative and the next transmitter (313) being selected at step 1308. Thus, these procedures repeat until all of the transmitter electrodes have been selected, resulting in the question asked at step 1308 being answered in the negative.

Thereafter, at step 1309, a question is asked as to whether another transmission set exists which, in this embodiment, on a first iteration, results in the second electrode set 312 being selected for transmission. Thus, the procedures are now repeated with the rolls of the electrodes reversed. In an alternative embodiment, only one iteration is performed if this is considered sufficient to provide an appropriate visual representation.

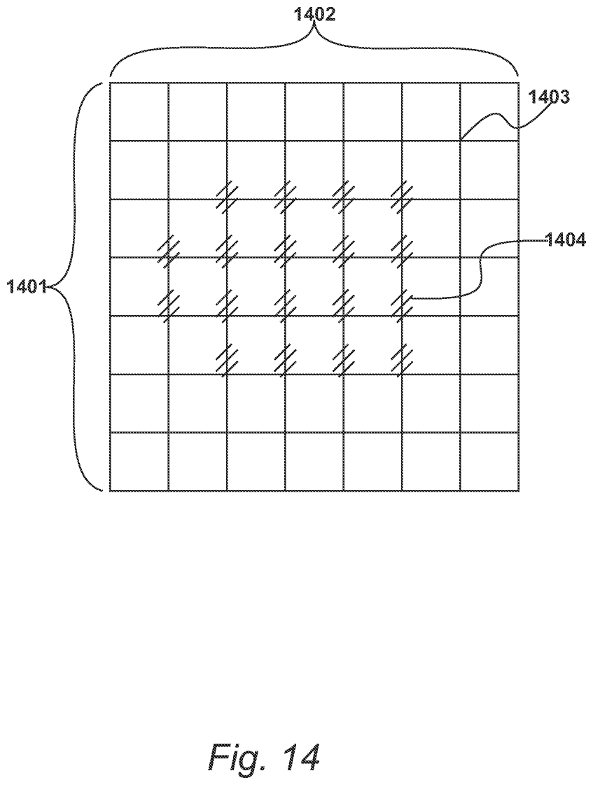

### FIG. 14

Following the procedures described with reference to FIG. 13, a visual representation is generated as illustrated in FIG. 14. A matrix is displayed of horizontal lines 1401 and vertical lines 1402, in accordance with the geometry of the scanning electrodes.

Following the procedure described with reference to FIG. 13, a data point has been collected for each intersection of these lines, such as intersection 1403. Thus, based on these results, an area surrounding each intersection, such as area 1404, becomes an illumination area; the illumination of which (or the shading of which) may be modified based on the results of the scanning operation.

### FIG. 15

As previously described with reference to FIG. 12, an electrode set is selected at step 1202 allowing forward layering to be performed at step 1203, followed by reverse layering at step 1204. These layering operations are performed in the horizontal direction, as indicated by arrow 342; with FIG. 15 showing an end view of the first electrode set 311. Thus, forward layering and reverse layering will be described with reference to the first electrode set 311; with similar procedures being performed (on the second iteration of the loop shown in FIG. 12) for the second electrode set 312.

Forward layering signals are produced, as identified at step 1203, using all eight of the available electrodes of the first electrode set 311, consisting of electrodes 321 to 328. Thus, the first upper electrode 321 is established as a transmitter electrode for seven strobing operations. This results in electrodes 322 to 328 being sequentially monitored and electric fields being generated, consisting of a first electric field 1501 to a seventh electric field 1507. As shown in FIG. 15, by increasing the displacement between the transmitter electrode and the receiver electrode, a greater level of penetration is achieved in the direction of arrow 1511.

In an embodiment, each electrode has a thickness of 0.5 millimetres and the distance between adjacent electrodes is 1.5 millimetres. Experiments show that an electric field will penetrate in the direction of arrow 1511 by a distance that is approximately half that of the distance between the transmitter and receiver electrodes. Thus, the spacing between the eight electrodes has been selected to ensure that appropriate levels of depth penetration are achieved, to allow signals to pass through the epidermis, the dermis and the lower fat layer of the location under examination. Furthermore, it is the signals that penetrate the dermis and the fat layer that are considered to be of the greatest importance.

If conditions are identified only with respect to the epidermis, these may be treated as not being life threatening and as such do not require further medical attention. Conditions requiring medical attention are identified when significant differences are monitored with respect to signals that do extend through the dermis and any subsequent layers.

### FIG. 16

Procedures 1204, for performing reverse layering, are illustrated in FIG. 16. This may be seen as performing the mirror image to forward layering as described with reference to FIG. 15. The combination of forward layering, followed by reverse layering as shown in FIG. 16, allows significant data to be obtained relating to various depths, without performing a very large number of strobing operations.

The strobing operations are performed in the direction of arrow 1601. The eighth electrode 328 is energized and the seventh electrode 327 is monitored, resulting in the generation of a first electric field 1611. The remaining electrodes are then sequentially selected, resulting in the generation of the second electric field 1612 to the seventh electric field 1617. Thus, in the embodiment, the electrodes are monitored first in ascending order as described with reference to FIG. 15 and then in descending order as described with reference to FIG. 16. However, it should be appreciated that, in other embodiments, the actual ordering of the unique strobing operations may vary.

### FIG. 17

Information derived from lower layers is crucially important in terms of providing information to the effect that a visible skin condition may or may not be malignant. The forward layering technique and the reverse layering technique, described with reference to FIGS. 15 and 16, provide substantial depth information, while optimizing the number of strobing cycles required. However, it should be appreciated that more depth information can be obtained if more strobing operations are performed within each scanning cycle.

As illustrated in FIG. 17, in addition to the first electrode 321 being energized followed by the eighth electrode 328 being energized, strobing operations may also energize other electrodes, such as the second electrode 322 and the third electrode 323. Ultimately, it is possible for a scanning cycle to include all possible combinations, such that each electrode is selected for energization seven times to interact with each remaining electrode as a receiver.

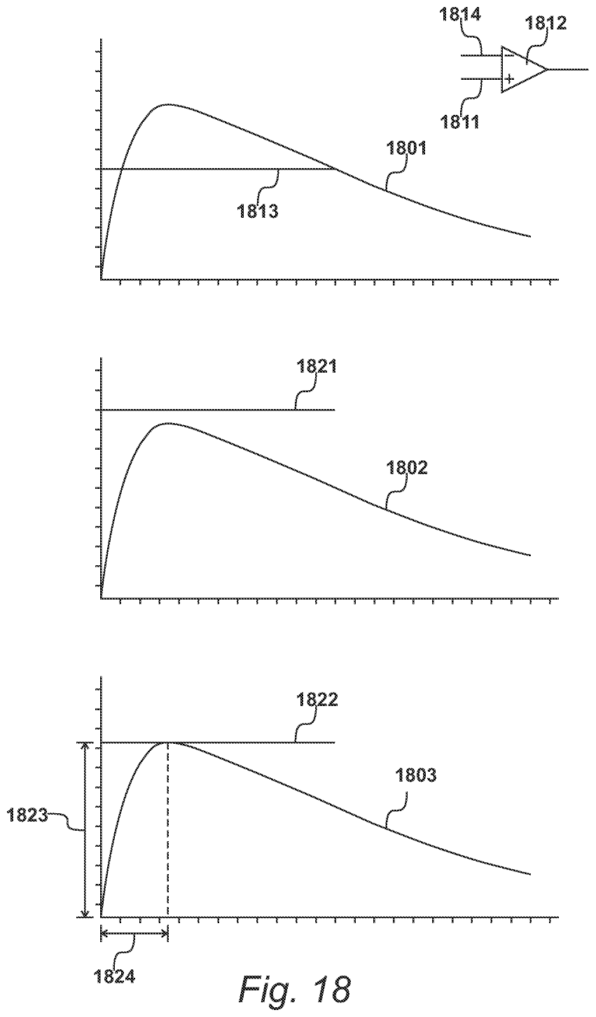

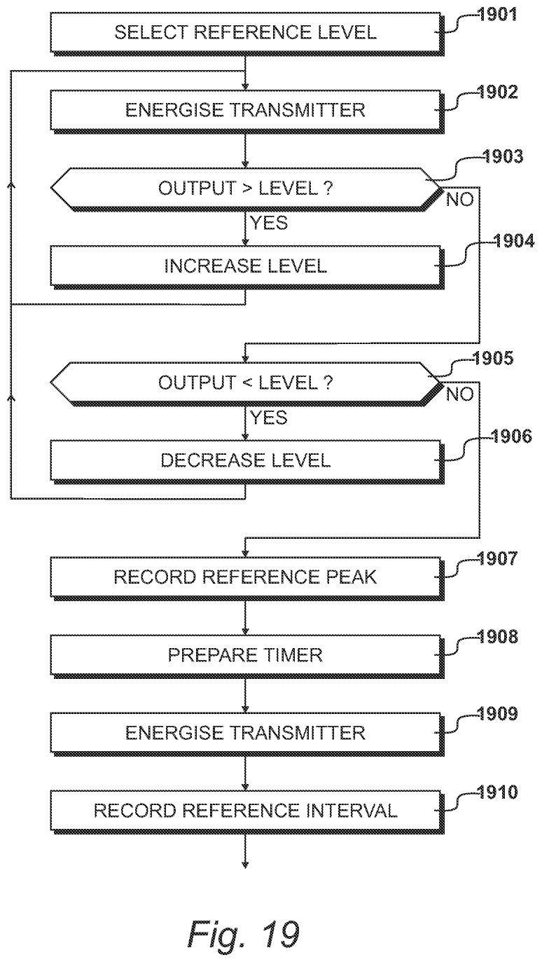

Whichever strobing operations are selected when scanning to produce reference signals, the same combinations (although not necessarily in the same order) are required when producing the test signals. However, similar selections do not undergo the same level of strobing. In particular, as described with reference to FIGS. 18 and 19, the generation of the reference signals requires plural strobing operations to be performed for each transmitter/receiver combination. However, when a similar scan is being performed to generate test signals, it is only necessary to energize a single strobing operation. Thus, in an embodiment, scanning to produce reference signals takes longer than scanning to produce test signals.

However, during the generation of the test signal data, a higher degree of sampling is performed in a first embodiment, although in an alternative embodiment, it is possible for the same degree of sampling to be performed with respect to the reference signals and the test signals.

### FIG. 18

Reference data is derived from reference output signals. Each reference output signal is produced by a single strobing operation. A first reference output signal 1801 is shown in FIG. 18, along with a second reference output signal 1802 and a third reference output signal 1803. These reference output signals have been produced for the same combination of transmitter electrode and receiver electrode and should therefore be substantially similar. For the purposes of this embodiment, they are considered to be substantially identical and may be viewed as multiple instantiations of the same electrical characteristic.

Multiple instantiations are required, generated by multiple strobing operations for the same combination of transmitter and receiver, because, in an embodiment, a reference peak value is identified for the reference output signal. In addition, a reference interval is determined for the reference output signal, in which the reference interval represents the duration from the start of an energizing pulse (i.e. the start of the strobing operation) to the reference peak value of the reference output signal. In an embodiment, the reference peak value is identified by comparing the output signal against a reference level, adjusting the reference level and repeating these steps until the reference level has been adjusted to the level of the reference peak value to be identified.

In an embodiment, the output signal, such as the first output signal 1801, is supplied to a positive input 1811 of a comparator 1812. In addition, a reference level 1813 is supplied to a negative input 1814 of the comparator 1812.

To an appropriate level of quantization, the reference peak value will be identified when a zero output is produced by the comparator 1812; in an embodiment, a latching circuit supplies the maximum value of the output signal to the positive input 1811 of the comparator 1812, such that the comparator 1812 compares the peak value of signal 1801 against the reference level 1813. If the result is negative, the reference level is increased to reference level 1821. In this example, it is compared with the second instance 1802 of the received signal, resulting in a negative output, given that the reference level is now higher.

The reference level is now reduced by a smaller amount and increments will continue to decrease (raising or lowering the reference level), until, given the level of quantization deployed, the reference level 1822 is equal to a reference peak value 1823.

In addition, a high frequency timer (generating a clock signal) is used to measure a reference interval, representing the time taken from the start of the strobing operation to the detection of the reference peak value 1823. The reference peak value 1823 and the reference interval 1824 represent, in an embodiment, reference data that has been derived from the reference output signals. To obtain this data, multiple strobing operations are required for each transmitter/receiver electrode pair but similar requirements are not necessary for the generation of test data.

### FIG. 19

Operations performed by the probe to achieve the functionality described with reference to FIG. 18 are shown in FIG. 19. At step 1901, a reference level (1813) is selected. A strobing operation is then initiated at step 1902 by energizing the transmitter electrode. This results in the generation of an output signal (1801) and at step 1903 the maximum value of the output signal is compared against the reference level selected at step 1901. Thus, at step 1903, a question is asked as to whether the maximum output level is greater than the reference level.

As shown at 1801, on the first iteration, the output level is greater than the reference level, therefore at step 1904 the reference level is increased.

On the next iteration, the transmitter electrode is energized again at step 1902 and again the question is asked at step 1903 as to whether the output maximum is larger than the reference level. On this occasion, as shown at 1802, the question asked at step 1903 is answered in the negative, such that, at step 1905, a question is asked as to whether the maximum output level is smaller than the reference level. On this second iteration, the reference level 1821 is larger, therefore the reference level is decreased at step 1906 and the transmitter is then energized again at step 1902, resulting in the generation of output reference signal 1803.

As described with reference to FIG. 18, the maximum level of the output reference signal 1803 is substantially the same as the adjusted reference level 1822, such that the question asked at step 1903 is answered in the negative and the question asked at step 1905 is answered in the negative. When these conditions are met, the reference peak level is recorded at step 1907. The timer is then prepared at step 1908 and a further energization of the transmitter occurs at step 1909. In this way, the reference interval is recorded at step 1910.

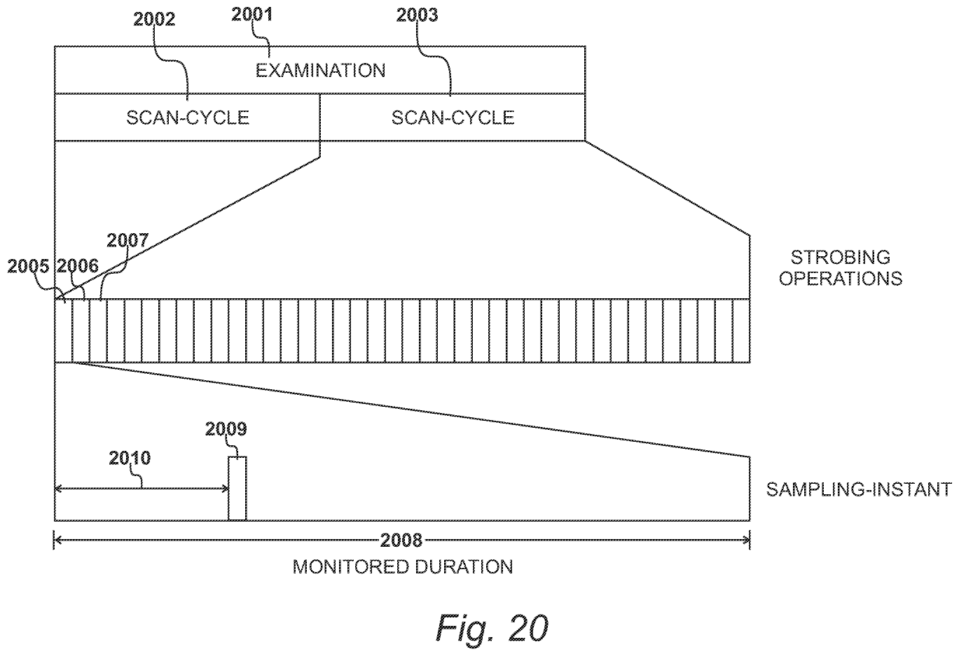

### FIG. 20

During a working period, many examinations may take place, each requiring an examination time 2001. During this examination time 2001, electrodes are energized sequentially during a two-dimensional scan cycle 2002 and during a forward and reverse layering scan cycle 2003.

During scan cycle 2002, many strobing operations are performed, including a first strobing operation 2005, a second strobing operation 2006, and a third strobing operation 2007 etc. An output signal, produced by capacitive coupling, is monitored and the process of generating test data from a monitored output test signal involves analog-to-digital conversion, thereby allowing the result of this conversion to be processed within the digital domain.

Each strobing operation 905 takes place within a monitored duration 2008. Within each monitored duration 2008, a sampling instant 2009 occurs, representing an instant within the monitored duration at which an output voltage is sampled.

In order to optimize results received from the examination process, the sampling instant does not occur immediately following the generation of an input strobing signal. Although, in an embodiment, a sharp, rapidly-raising strobing input signal is supplied to the transmitters, the shape of resulting output signals will not rise so steeply; as a result of the electrical properties of the device and the electrical properties of the biological material being scanned.

In previous applications of scanning technology of this type, the sampling instant 2009 is delayed by a delay period 2010. However, in previous systems, the delay period was predetermined and thereafter fixed. In some situations, as could be used in an embodiment, the delay period attempted to delay sampling until the peak of the output signal. However, the present invention seeks to improve accuracy by comparing test data against reference data. In an embodiment, this is achieved by identifying the peak of the reference signal, measuring the interval at which this peak occurs and then using this measured interval as the delay period 2010 when obtaining test data.

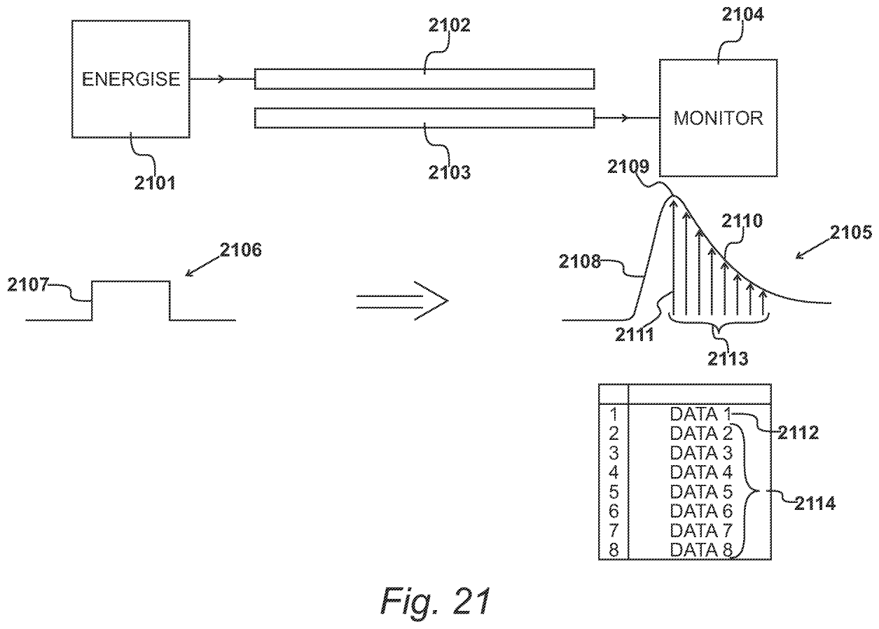

### FIG. 21

The sampling of a test output signal is illustrated in FIG. 21. An energizing process is illustrated at 2101 that energizes a transmitter electrode 2102 during a strobing operation. During this strobing operation, a receiver electrode 2103 is monitored, as illustrated at 2104, to produce an analog output signal 2105. A strobing pulse 2106 includes a sharp rising edge 2107 but with the receiver electrode 2103 being capacitively coupled to the transmitter electrode 2102, the resulting analog output signal has characteristics determined by the impedance of the transmission environment. This results in the presentation of a rising slope 2108, a peak-value 2109 and a falling slope 2110.

The analog output signal is sampled, as illustrated by a first arrow 2111 to produce first sample data 2112. In addition, further sampling 2113 is performed to produce additional sample data 2114 during the strobing operation.

In an embodiment, multiple samples of test signals are taken. However, in preference to estimating the position of the peak of each test signal, the first sampling position occurs at the position of the peak of the reference signal; thereby optimizing comparisons between reference data and test data for similar strobing operations.

Further sampling steps produce additional sample data after the first sample. In the embodiment illustrated in FIG. 21, the further sampling step produces seven instances (DATA 2-DATA 8) of additional sample data 2114.

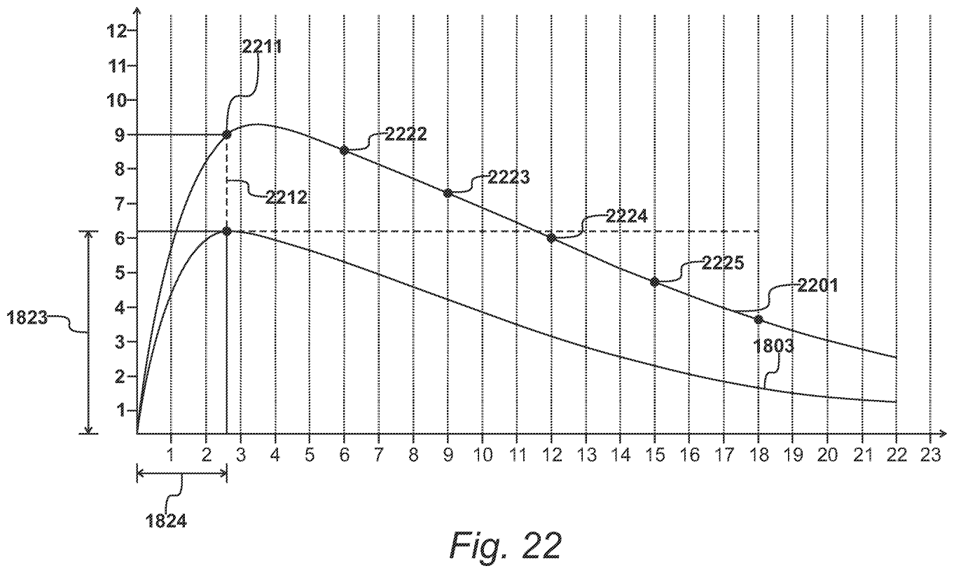

### FIG. 22

A strobing operation produces an output signal. The present invention requires output signals and test signals to be produced. Many signals of this type are produced for different combinations of transmitter and electrode pairs, such that, for each pair, a reference signal and a test signal are obtained. Reference signals are produced when the probe has been deployed against a region of skin that does not include the skin condition. Similarly, the probe is then redeployed to produce test signals when located at the position of the visible skin condition. For the same electrode combinations, test data is compared with reference data, each derived from their respective signals.

An example of reference output signals was described with reference to FIG. 18 and reference output signal 1803 is shown in FIG. 22. From this, reference data is derived, as previously described with reference to FIG. 18 and FIG. 19, in the form of the reference peak value 1823 and a reference interval 1824. This data is derived at step 1103, previously described with reference to FIG. 11.

Test signals are produced at step 1105, including a test signal 2201 generated from the same electrode combination as that used for the generation of reference signal 1803. Having generated the test signal, test data is derived at step 1106. In an embodiment, test data is obtained by sampling a first test value 2211 after the reference interval 1824. Thus, the reference interval 1824, calculated from the reference signal, is used to create a sampling instant for the test signal, as a mechanism for generating the test data. It is then possible to compare the first test value 2211 with the reference peak value 1823 to identify the difference between these values, as shown at 2212.

Furthermore, the greater the size of difference value 2212, the greater the likelihood of the region under consideration requiring medical attention. In addition, this requirement is enhanced further when the degree of penetration is greater. Thus, a difference value 2212 has greater significance when derived from, say, electric field 1617 compared to electric field 1611. The procedure, in this embodiment, is therefore looking for difference values of significant size that occur with respect to electric fields penetrating a significant depth. In an embodiment, a threshold may be established for comparing the product of the first test value with the depth of penetration.

When sampling output signals of the type shown in FIG. 22, it is known to have a fixed sampling instant that is present for the particular application concerned. The present embodiment introduces a higher level of sophistication, in that the sampling instant for the test signal 2201 is derived from an analysis of the reference signal and, in particular, is based on the reference interval 1824, which is itself determined from the reference peak value 1823.

In this embodiment, a second test value 2222 is sampled, along with a third test value 2223, a fourth test value 2224 and a fifth text value 2225. Thus, in this embodiment, five sample points are derived from each test output signal although, in alternative embodiments, more or fewer sample points could be obtained.

In an embodiment, it would be possible to determine the position of the second to the fifth sampling points, again with respect to the reference interval 1824. Thus, in an embodiment, further samples could be taken at multiples of the reference interval 1824. In the embodiment of FIG. 22, the second to the fifth test values are taken at predetermined sampling points which, in this example, occur after six microseconds, nine microseconds, twelve microseconds and fifteen microseconds. In this embodiment, each of the second to the fifth test values are compared against the reference peak value 1823.

In an embodiment, test data is obtained by assessing the number of test values that are above the reference peak value and the number that are below. In the example shown in FIG. 22, the second test value 2222 and the third test value 2223 are both above the reference peak value 1823. Similarly, the fourth test value 2234 and the fifth test value 2225 are below the reference peak value 1823. Thus, in an embodiment, the test data, obtained from the test output signals, may consist of an identification of the difference value 2212 followed by an indication as to when the test signal drops below the reference peak value. In this embodiment, the data set would include an identification of the third test value and the fourth test value as the ones laying just above and just below the reference peak value.

In an alternative embodiment, multiple samples are also taken for the reference signal 1803, such that specific comparisons may be made at the same instances, possibly at six microseconds, nine microseconds, twelve microseconds and fifteen microseconds.

The invention claimed is:

1. An apparatus for producing output data indicative of a visible skin condition requiring medical attention, comprising:
a manually-deployable probe; and
a data processing system, wherein:
said manually-deployable probe includes a plurality of electrodes, a strobing circuit and a monitoring circuit;
said monitoring circuit monitors output signals from a selected output electrode of said plurality of electrodes produced by capacitive coupling when a selected input electrode of said plurality of electrodes receives an energizing strobing signal; and
said data processing system is configured to:
operate said manually-deployable probe to produce reference output signals;
derive reference data from said reference output signals;
operate said manually-deployable probe to produce test output signals;
derive test data from said test output signals; and
compare said test data against said reference data to produce said output data.

2. The apparatus of claim 1, wherein said manually-deployable probe includes a visual indicator, to indicate:
an availability to be deployed;
reference strobing; and
test strobing.

3. The apparatus of claim 2, wherein said manually-deployable probe includes a manually operable device for indicating a start of said reference strobing and a start of said test strobing.

4. The apparatus of claim 1, wherein said data processing system is configured to derive said reference data by:
identifying a reference peak value for a reference output signal of said reference output signals; and
determining a reference interval for said reference output signal, wherein:
said reference interval represents a duration from a start of an energizing pulse to said reference peak value.

5. The apparatus of claim 4, wherein said reference peak value is identified by:
comparing an output signal of said output signals against a reference level;
adjusting said reference level; and
repeating said comparing step and said adjusting step.

6. The apparatus of claim 4, wherein:
said reference interval is determined by measuring an interval from said start of said energizing pulse to said reference peak value with reference to a clock signal.

7. The apparatus of claim 4, wherein:
said test data is obtained by sampling a first test value after said reference interval, from a start of a strobing operation, such that
said output data is produced by comparing said first test value against said reference peak value.

8. The apparatus of claim 7, wherein said first test value is compared with said reference peak value by:
subtracting a modulus of said reference peak value from a modulus of said first test value to produce a difference value; and
producing an output signal indicative of a requirement for medical attention if said difference value is positive.

9. The apparatus of claim 4, wherein said data processing system is configured to:
further sample a test output signal of said test output signals after said reference interval to produce further test samples; and
compare said further test samples against said reference peak value.

10. A method of producing output data indicative of a visible skin condition requiring medical attention, comprising the steps of:
identifying a reference region of skin that does not include said visible skin condition;
deploying a probe against said reference region to produce reference data; and
re-deploying said probe against said visible skin condition to produce test data, wherein:
output electrodes of said probe produce output signals by capacitive coupling when input electrodes of said probe receive energizing pulses;
said reference data is derived from reference output signals;
said test data is obtained from test output signals; and
said output data is produced by comparing said test data against said reference data.

11. The method of claim 10, wherein said reference data is derived by the steps of:
identifying a reference peak value for a reference output signal of said reference output signals; and
determining a reference interval for said reference output signal, wherein:
said reference interval represents a duration from a start of an energizing pulse of said energizing pulses to said reference peak value.

12. The method of claim 11, wherein said reference peak value is identified by:
comparing an output signal of said output signals against a reference level;
adjusting said reference level; and
repeating said comparing step and said adjusting step.

13. The method of claim 11, wherein:
said reference interval is determined by measuring an interval from said start of said energizing pulse to said reference peak value with reference to a clock signal.

14. The method of claim 11, wherein:
said test data is obtained by sampling a first test value after said reference interval, from a start of a strobing operation, such that
said output data is produced by comparing said first test value against said reference peak value.

15. The method of claim 14, wherein said step of comparing said first test value with said reference peak value comprises the steps of:
subtracting a modulus of said reference peak value from a modulus of said first test value to produce a difference value; and
producing an output signal indicative of a requirement for medical attention if said difference value is positive.

16. The method of claim 10, wherein said capacitive coupling produces output signals influenced by a permittivity and a conductivity of electric-field-penetrated tissue.

17. The method of claim 11, further comprising the steps of:
further sampling a test output signal of said test output signals after said reference interval to produce further test samples; and
comparing said further test samples against said reference peak value.

18. The method of claim 17, wherein said further test samples consists of four further test samples to give a total of five sample points for each test-data strobing operation.

19. The method of claim 17, wherein:
an originating test output signal is modelled from said further test samples to produce a test-signal model; and
a test-data peak level is identified from said test-signal model.

20. The method of claim 10, wherein:
said step of deploying a probe against said reference region to produce reference data includes performing plural strobing operations with respect to selected combinations of transmitter electrodes and receiver electrodes; and
said step of re-deploying said probe against said visible skin condition to produce test data includes performing substantially similar strobing operations to said step of deploying a probe against said reference region to produce reference data, using said selected combinations of transmitter electrodes and receiver electrodes.
